(19) United States
(12) Patent Application Publication
(10) Pub. No.: US 2007/0225670 A1
(43) Pub. Date: Sep. 27, 2007

(54) Diapers for improving male genital health and methods of using the same

(76) Inventor: Thomas Jennings Connell, Vienna, VA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/724,019

(22) Filed: Mar. 14, 2007

Related U.S. Application Data
(60) Provisional application No. 60/782,140, filed on Mar. 14, 2006.

(51) Int. Cl. A61F 13/15

(52) U.S. Cl. 604/385.09; 604/385.19

(57) ABSTRACT

A diaper and method for improving male genital health, the diaper including a non-absorbent insert positioned on an inner surface of an inner layer in the proximity of a front region thereof such that when the diaper is worn by a male wearer the insert is positioned adjacent the penis of the male wearer. In the method, a non-absorbent insert is interposed between the meatus and the absorbent diaper to prevent undesired drying of the meatus which can lead to meatal stenosis and related complications. In a further aspect of the diaper and method, the insert may be coated or impregnated with moisturizing agents and/or therapeutic agents.

### CROSS-REFERENCE TO RELATED APPLICATION

The present application claims the benefit of and priority to U.S. Provisional Application Ser. No. 60/782,140, filed on Mar. 14, 2006, the entire content of which is incorporated herein by reference.

### BACKGROUND

1. Technical Field

The present disclosure relates generally to diapers, and, more particularly, to diapers having a non-absorbent insert positioned to separate the genitalia of the male wearer from the absorbent diaper.

2. Background of Related Art

Disposable diapers and the like are well known in the art. Disposable diapers are available in sizes to fit infants through adults, and may be especially adapted for male or female use. Regardless of the intended wearer, disposable diapers function to absorb the fluid discharge of the wearer. Modern diapers generally consist of multiple layers, each acting together to prevent leakage outside the diaper, but also keeping the skin of the wearer dry. Unfortunately, the absorption capability of diapers and the placement of the absorbent layers within the diaper can lead to serious complications in the developing genitalia of male wearers.

Meatal stenosis is a complication that can develop as a result of the use of absorbent diapers with male wearers. The urethra is a small tube through which urine passes from the bladder. The opening at the end of the urethra where urine leaves the penis is called the meatus which is normally found at the tip of the penis. Meatal stenosis is the name given to the narrowing of this opening. The narrowing of the meatus essentially causes backpressure to build up during urination, which in turn causes the urethra to stretch and/or radially expand under the strain of the fluid pressure. The stretching and/or radial expansion of the urethra may cause discomfort to the patient, which in turn may cause the patient to resist or slow urination. The condition is characterized by frequent urination at low flow rates which appears as a smaller stream of flow than normally would be observed. In addition, the condition may cause an upward deflection of the weakened urine stream due to the fact that the scarring that causes the narrowing of the meatus is more prominent on the ventral side of the meatus. Although rare, recurrent urinary tract infections may also result from this condition.

Meatal stenosis can be caused by repeated irritation to the tip of the penis. Uncircumcised males are less likely to develop meatal stenosis because of the protection provided by the foreskin. In a circumcised penis, where the meatus is exposed, exposure to wet and abrasive materials such as a diaper can cause irritation to tissues forming the meatus. The continued irritation can cause scar tissue to begin to develop. The developing scar tissue may continue to constrict the opening as conditions persist. The resulting scar tissue is more rigid and less flexible than the natural healthy tissue, and results in a constricted opening, which does not enlarge as the child grows.

In the past, in an attempt to prevent irritation caused by wet diapers, an absorbent material was added to the diaper that would draw the wetness away from the body. The absorbent materials currently used in disposable diapers are extremely effective at absorbing fluids. Unfortunately, while the diaper absorbs the wearer's discharge, the absorbent diaper also dries out the tissue forming the meatus. Repeated desiccation of the tissue forming the meatus by an absorbent diaper can cause scar tissue to develop. Accordingly, continued exposure of the penis to the absorbent diaper may lead to meatal stenosis.

Phimosis, a condition characterized by the inability to retract the foreskin of an uncircumcised penis over the penis head, or glans, may also be attributable to the excessive drying of the penile tissues, the meatus, etc.

Therefore, it would be desirable to have a diaper including an insert separating the genitalia of a male wearer from the absorbent material thereof in order to reduce the incidents of meatal stenosis and the like.

### SUMMARY

A diaper for improving male genital health is provided. The diaper includes a front region and a back region, the front region corresponding to a front of a male wearer, and the back region corresponding to a back of the male wearer. The diaper further includes an outer liquid impermeable layer having a length; an inner absorbent layer overlying and extending for at least a portion of the length of the outer layer. The inner layer has an inner surface, a front region, a central region, and a back region, wherein the front region of the inner layer corresponds to the front region of the diaper; and a non-absorbent insert positioned on the inner surface of the inner layer in the proximity of the front region thereof. Accordingly, when the diaper is worn by a male wearer the insert is positioned adjacent the penis of the male wearer.

The diaper may further include at least one fastener connected to the outer layer for securing the back region of the diaper to the front region of the diaper when the diaper is worn by the male wearer.

The insert may be attached to the inside surface of the inner layer with an adhesive. The insert may be selectively attachable to the inside surface of the inner layer or the insert may be integrally formed into the inner layer.

The diaper may further include a band of absorbent material extending about at least a portion of the perimeter of the insert. The insert is at least one of circular, rectangular, triangular, and trapezoidal in shape. The insert may form a pocket that can be triangular or trapezoidal in shape. The pocket may be selectively attachable to the inside surface of the inner layer or the pocket may be integrally formed into the inner layer.

The insert may include a moisturizing agent. Exemplary moisturizing agents include zinc oxide and petroleum jelly.

According to another aspect of the present disclosure, a method of improving male genital health is provided. The method includes the step of interposing a non-absorbent layer between the meatus and a diaper.

It is envisioned that the non-absorbent layer may be pre-attached to the diaper. The non-absorbent layer may be a pocket pre-attached to the diaper.

The method may further include the step of providing a moisturizing agent associated with the non-absorbent layer. The method may still further include the step of providing a therapeutic agent associated with the non-absorbent layer.

According to a further aspect of the present disclosure, an insert for use in combination with a diaper having an inner absorbent layer is provided. The insert includes a liquid impermeable layer selectively affixable to the inner absorbent layer of the diaper. The insert is positioned on the inner layer of the diaper at a location in close proximity to the meatus of a penis when the diaper is worn by a patient.

The insert may further include a securing means for selectively affixing the insert to the inner layer of the diaper. The securing means may be selected from the group consisting of tape, adhesives, stitches, and hook and loop fasteners.

The insert may further include a moisturizing agent at least one of impregnated in the non-absorbent layer and coated on the non-absorbent layer.

The insert may be in the form of a pocket configured and dimensioned to receive the meatus of the penis therein.

The insert may be at least one of circular, ovular, rectangular and triangular.

The insert may further include an absorbent border extending at least partially around a perimeter thereof.

### BRIEF DESCRIPTION OF THE DRAWINGS

Preferred embodiments of the present disclosure will be better appreciated by reference to the drawings wherein:

FIG. 1 is a plan view of the inside surface of a prior art disposable diaper;

FIG. 1A is an enlarged sectional view of the disposable diaper of FIG. 1 taken along line 1A-1A thereof;

FIG. 2 is a plan view of the inside surface of a diaper according to one embodiment of the present disclosure;

FIG. 2A is an enlarged sectional view of the disposable diaper of FIG. 2 taken along line 2A-2A thereof;

FIG. 3 is a plan view of the inside surface of a diaper according to another embodiment of the present disclosure;

FIG. 3A is an enlarged sectional view of the disposable diaper of FIG. 3 taken along line 3A-3A thereof;

FIG. 4 is a plan view of the inside surface of a diaper according to still another embodiment of the present disclosure;

FIG. 4A is an enlarged sectional view of the disposable diaper of FIG. 4 taken along line 4A-4A thereof;

FIG. 5 is a plan view of the inside surface of a diaper according to yet another embodiment of the present disclosure;

FIG. 5A is an enlarged sectional view of the disposable diaper of FIG. 5 taken along line 5A-5A thereof;

FIG. 6 is a plan view of the inside surface of a diaper according to an embodiment of the present disclosure; and

FIG. 6A is an enlarged sectional view of the disposable diaper of FIG. 6 taken along line 6A-6A thereof.

### DESCRIPTION OF EMBODIMENTS

Embodiments of the presently disclosed diapers for improved male health will now be described in detail with reference to the drawing figures in which like reference numerals designate identical or corresponding elements in each of the several views.

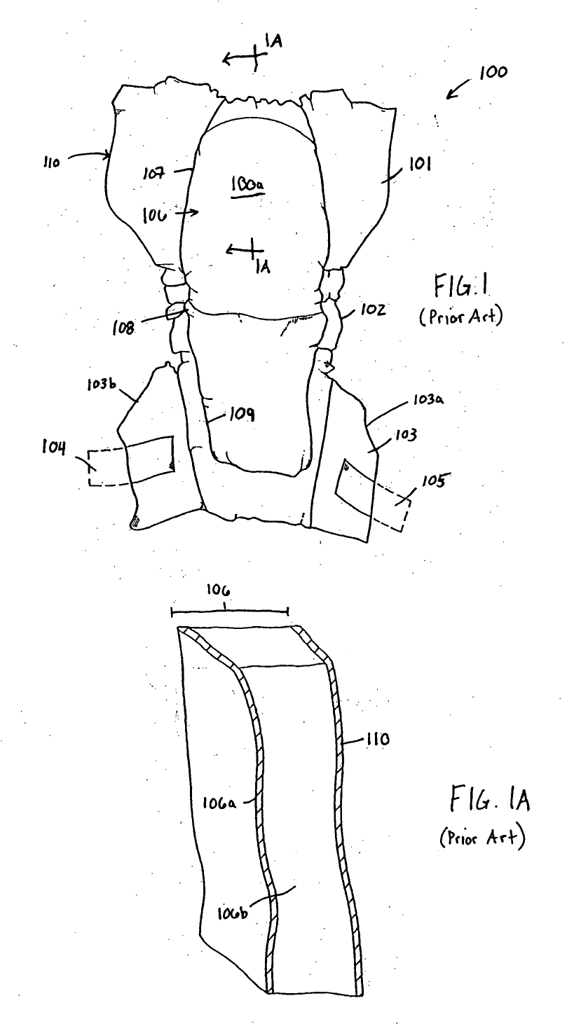

Referring initially to FIGS. 1 and 1A, an inside surface 100a of a conventional prior art disposable diaper 100 is shown. Diaper 100 includes a generally rectangular shaped liquid impermeable outer layer 110 having a front region 101, a central region 102, and a back region 103. The long sides of outer layer 110 are preferably concave in the vicinity of central region 102, in order to comfortably accommodate the upper inside thigh of the wearer.

Diaper 100 also includes an absorbent inner layer 106 having a generally rectangular shape. Inner layer 106 is typically adhered to liquid impermeable outer layer 110 by means known by one having skill in the art. Inner layer 106 includes a front region 107, a central region 108 and back region 109 overlying and in juxtaposed relationship to front region 101, central region 102, and back region 103 of the liquid impermeable outer layer 110, respectively. In the embodiment shown in FIG. 1 and 1A, inner layer 106 includes a liquid permeable layer 106a and an absorbent layer 106b overlying permeable layer 106a. Alternately, inner layer 106a may include a liquid absorbent core completely enclosed by cloth or other liquid permeable material. The inner layer 106a may also be fabricated entirely of a single absorbent layer or thick padding of material.

Diaper 100 includes tabs 104, 105 extending from respective opposite sides 103a, 103b of back region 103. Tabs 104, 105 are used to attach the back region 103 of outer layer 110 to front region 101 when diaper 100 is worn in a usual, recommended, or proper manner. Tabs 104, 105 include an adhesive for securely adhering to diaper 100. Tabs 104, 105 may also be secured with an eye and hook fastener, such as VELCRO®, available from Velcro Industries B.V.

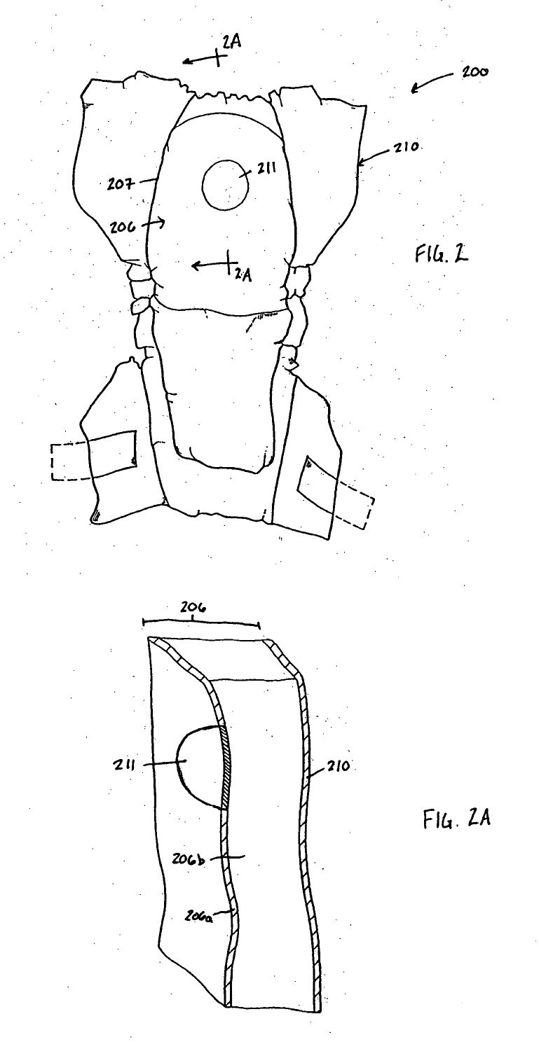

Referring now to FIGS. 2 and 2A, a diaper in accordance with an embodiment of the present disclosure is illustrated and is designated as reference number 200. Diaper 200 includes an outer layer 210, an absorbent inner layer 206, and an insert 211 for protecting the penis of the male wearer from contacting absorbent inner layer 206 of diaper 200. Absorbent inner layer 206 includes a liquid permeable layer 206a and an absorbent layer 206b interposed between liquid permeable layer 206a and outer layer 210. In the present embodiment, insert 211 is integrally formed within liquid permeable layer 206a of inner layer 206.

As seen in FIG. 2, insert 211 is positioned on front region 207 of inner layer 206 such that when the diaper is worn by a male wearer, insert 211 is located adjacent to and or in contact with the penis. Insert 211 corresponds in size and shape to the region of the diaper in which the penis of the male wearer will contact the diaper. As seen in FIGS. 2 and 2A, insert 211 may have a generally circular profile. However, it is within the scope of the present disclosure for insert 211 to have any shaped profile, including and not limited to rectangular, ovular, triangular, etc. The placement of insert 211 on or in absorbent inner layer 206a can be modified and/or located thereon or therein in accordance with the dimensions of the wearer. In accordance with the present disclosure, use or inclusion of an insert 211 that covers more of the absorbent area than the penis can contact is unnecessary and detracts from the effectiveness of the disposable diaper in controlling wetness. In an embodiment, insert 211 is sized to be no larger than the area in which the penis of the male wearer contacts inner layer 206.

As shown in FIG. 2A, insert 211 may have substantially the same thickness as liquid permeable layer 206a. However, it is understood and within the scope of the present disclosure for insert 211 to also be thicker or thinner than liquid permeable layer 206a. When insert 211 is thicker than liquid permeable layer 206a insert 211 may extend beyond the surface of the liquid permeable layer 206a in either or both directions (i.e., internally or externally). Insert 211 can be affixed to liquid permeable layer 206a by any known fastening and/or securing means, including and not limited to tape, adhesive, stitching, hook and loop fasteners, etc.

Insert 211 is fabricated from a non-absorbent material to prevent moisture from being wicked from the tissue forming the meatus of the penis. In an embodiment, the material forming insert 211 may also be non-abrasive. Insert 211 may also be coated or impregnated with a moistening or moisturizing agent. The moistening or moisturizing agent can help to reduce irritation of the tissues of the penis as well as to help prevent the tissues from drying out. Common moistening or moisturizing agents include ointments containing zinc oxide, sold under the tradename DESITIN® by Warner-Lambert Company, LLC, a Pfizer company, and petroleum jelly, sold under the tradename VASELINE® by Unilever, Inc.

In addition to or in place of moisturizing agents, the insert may be coated or impregnated with a therapeutic agent to enhance wound healing and/or prevent infection. Exemplary therapeutic agents may include and are not limited to Antibiotics (e.g., Bacitracin, Neosporin or other topical antibacterial or bacteriocidal agents; and/or wound healing agents (e.g., Zinc Oxide, Vitamin E, Balmex, Desitin, Aloe, Aquaphor, A and D ointment). In the case of either moisturizing or therapeutic agents, the insert may release the moisturizing agent or therapeutic agent in a time release fashion during the time the diaper is worn, e.g., several hours. As will be appreciated, the inclusion of moisturizing agents or therapeutic agents can be applied to any embodiment described herein.

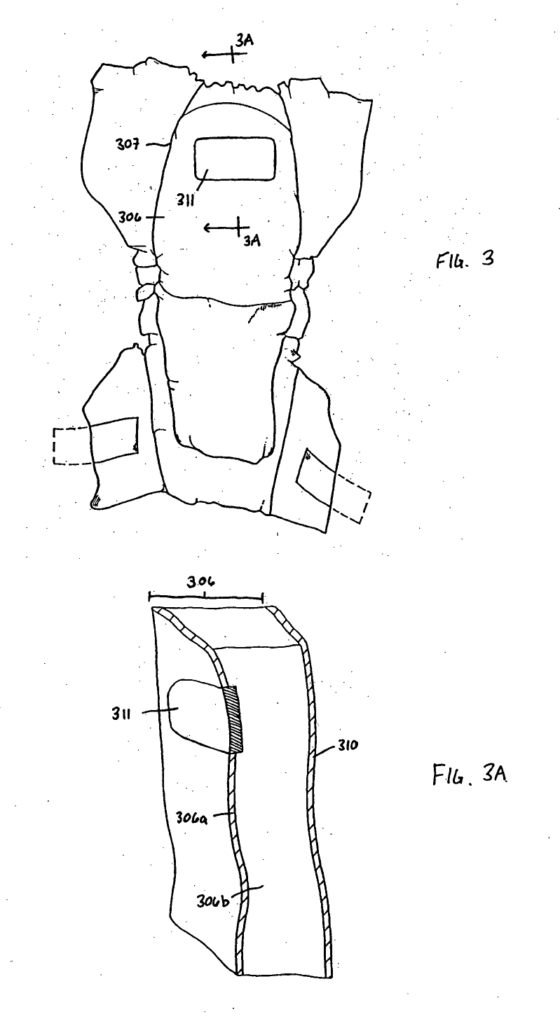

Referring now to FIGS. 3 and 3A, an alternate embodiment of diaper 200 is generally shown as 300. Diaper 300 is similar to diaper 200 and thus will only be discussed in detail herein to the extent necessary to identify differences in construction and operation.

As seen in FIGS. 3 and 3A, diaper 300 includes an insert 311 placed on and/or in absorbent layer 306a having a substantially rectangular shape. Although insert 311 is illustrated in FIGS. 3 and 3A as being oriented in a transverse direction, rectangular insert 311 may be oriented in any direction and be of any size. Preferably, insert 311 is oriented and sized so as to cover, or replace, the area of the absorbent diaper that the penis of the male wearer will contact.

As seen in FIG. 3A, insert 311 is integrally formed with liquid permeable layer 306a of front region 307 and extends into absorbent layer 306b. As is contemplated herein, use of a thicker insert 311 offers more protection for the penis from the absorbent layers of the diaper. Also, use of a thicker insert 311 allows for the possibility of using or including additional moistening or moisturizing agents, or similar solutions.

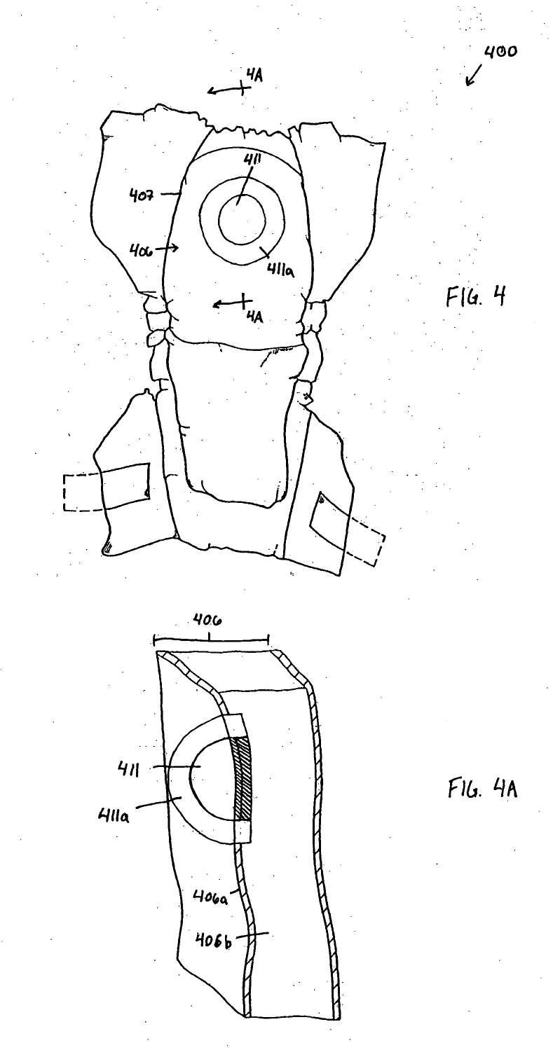

Referring now to FIGS. 4 and 4A, an alternate embodiment of diaper 200 is generally shown as 400. Diaper 400 is substantially similar to diaper 200 with the exception that insert 411 is constructed with an outer absorbent band or border 411a. Absorbent border 411a extends radially outward from insert 411 so as to receive any fluid discharge which is diverted from insert 411. Absorbent border 411a may be constructed from a material identical to or substantially similar to the absorbent material found in disposable diaper 400. It is envisioned that absorbent border 411a may also be more absorbent then the absorbent material 406b of diaper 400. Inclusion of an absorbent border 411a around insert 411 helps to compensate for the surface area and/or volume of absorbable material 406 lost to inclusion of insert 411. Absorbent border 411a may completely encircle the circumference of insert 411, or any portion of the circumference of insert 411. Insert 411 and absorbent border 411a are integrally formed onto or into liquid permeable layer 406a of front layer 407 of diaper 400. As with prior disclosed embodiments, insert 411 and absorbent border 411a may be affixed to diaper 400 with any known means, including and not limited to adhesives, stitching, tape or the like.

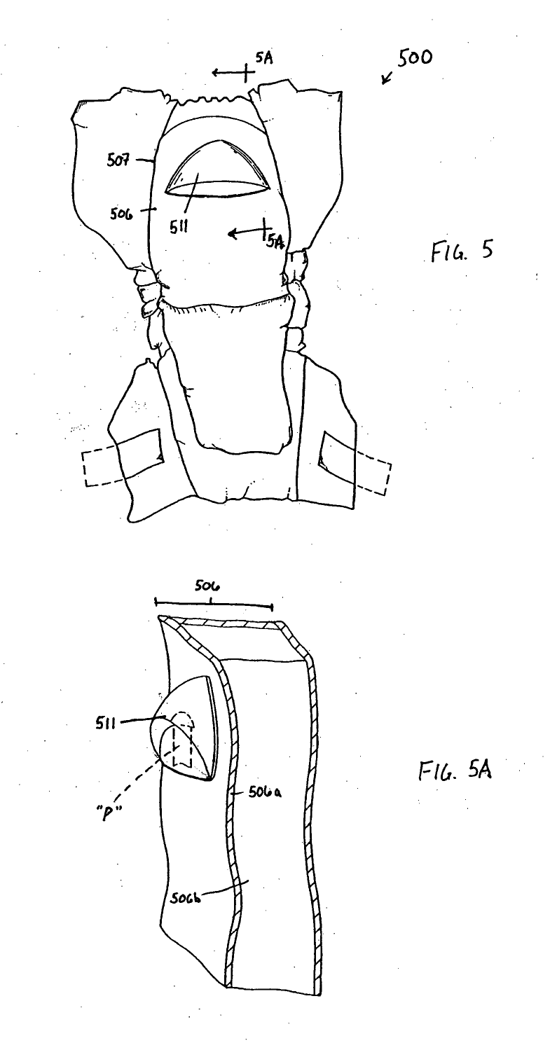

Referring now to FIGS. 5 and 5A, yet another embodiment of diaper 200 is shown and is generally designated as 500. Diaper 500 is substantially similar to diaper 200 with the exception insert 511 is in the form of a pouch or pocket. Desirably, insert 511 is substantially hemispherical in shape. Insert 511 is made from a non-absorbent material and is, preferably, non-abrasive. Insert 511 is sized and positioned on permeable layer 506a of front region 507 so as to accept the penis “P” of the male wearer. Insert 511 is configured and adapted to prevent the meatus of the penis “P” from contacting absorbent material 506b of diaper 500. Insert 511 may be integrally formed with liquid permeable layer 506a of inner layer 506 in front region 507 of diaper 500. Alternatively, insert 511 may be selectively positionable on liquid permeable layer 506a of inner layer 506. As with prior disclosed embodiments, insert 511 be affixed to diaper 500 with any known means, including and not limited to adhesives, stitching, tape or the like.

In addition to separating the penis “P” of the wearer from the absorbent material of the diaper, insert 511 acts to deflect the upward stream of urine that is often exhibited in patients suffering from meatal stenosis. As explained above, the scar tissue causing the narrowing of the opening is more prominent on the ventral side of the meatus. As a result, the stream of urine is discharged upwardly from the penis along the body. Because the absorbent sections of most disposable diapers are located at or below the penis, without insert 511 the upwardly deflected stream of urine could render the diaper useless and/or less efficient.

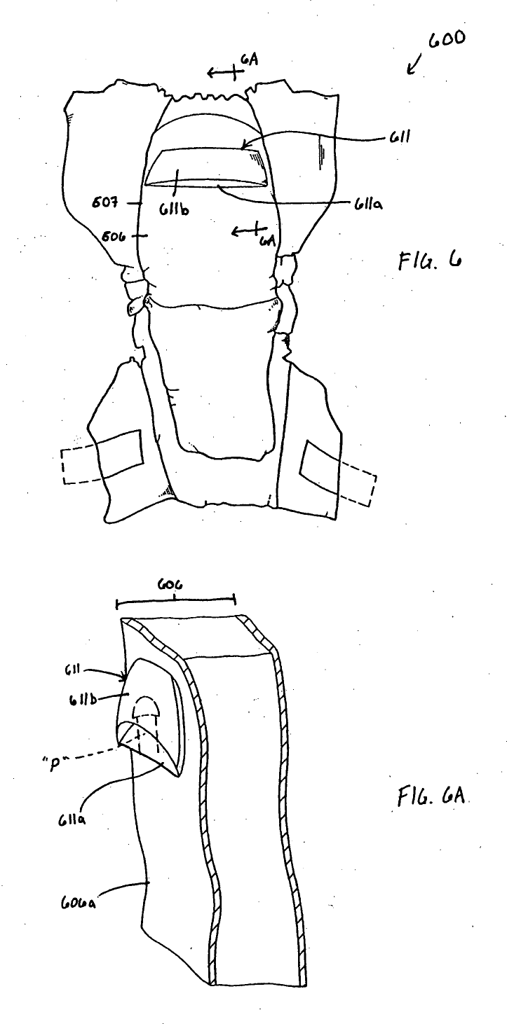

Turning now to FIGS. 6 and 6A, another embodiment of diaper 500 is generally designated as diaper 600. Here, diaper 600 includes an insert 611 in the form of a pouch or pocket. Insert 611 has a substantially trapezoidal shape. A lower portion 611a of insert 611 may be secured to permeable layer 606a of front region 607 with an adhesive, tape or the like and an upper portion 611b extending over lower portion 611a. Insert 611 may also be integrally formed with inner layer 606 of diaper 600. Like insert 511, insert 611 is designed to prevent the upward deflected stream of urine from exiting the diaper. Insert 611 is composed of a non-absorbent material. In alternate embodiments, insert 611 may have an absorbent outer layer and a protective, non-absorbent inner layer.

It is envisioned that any of the inserts disclosed hereinabove may be selectively positionable on the inner surface of the diaper. A selectively positionable insert allows for better alignment and positioning of the insert with respect to the penis of the wearer. A selectively positionable insert may be affixed to an inner front surface of the diaper with an adhesive, tape, VELCRO® and the like. As with the integrated insert, the selectively positionable insert may take the form of any shape (e.g., circular, ovular, triangular, rectangular, etc.). Preferably the size and shape of the insert corresponds to the surface area in which the penis of the wearer will contact the inner surface of the diaper. The selectively positionable insert may also be coated or impregnated with a moistening or moisturizing agent. The selectively positionable insert can be designed for use with all diapers including disposable and reusable cloth diapers. The selectively positionable insert itself may be disposable or reusable. The selectively positionable insert may also contain an absorbent border extending therearound, as substantially show in FIGS. 4 and 4A. In yet a further alternative, the protective layer or pocket may be placed adjacent or over the penis during diaper changing without being attached to the diaper.

As indicated above, the present disclosure is directed to providing a means for separating the penis of a wearer from the absorbent material of a disposable diaper. While the present disclosure has been particularly shown and described with reference to preferred embodiments thereof, it will be understood by those skilled in the art that various changes in form and details may be made therein without departing from the spirit and scope of the invention.

In accordance with the method of using the disclosed apparatus, in order to prevent or reduce the incidence of meatal stenosis or similar conditions, a non-absorbent material is disposed between the meatal opening of the penis and a diaper, such as after circumcision. In accordance with one embodiment of the method, the non-absorbent material is a sheet or layer attached to the front inside portion of the diaper, such as described in connection with FIGS. 2-2A, 3-3A or 4-4A, adjacent the penis to eliminate or reduce contact between the meatus and the absorbent diaper. Thus, the method in the simplest form consists of interposing a non-absorbent material between the meatus of a penis and a diaper. In a second embodiment of the method, the non-absorbent material takes the form of a pocket, such as described in connection with the embodiments of FIGS. 5-5A and 6-6A. The non-absorbent material may be a sheet or pocket which separates the meatus from the absorbent diaper, thereby protecting the meatus from drying effects of the absorbent diaper that can cause drying, irritation and scarring of the tissue in and around the meatus. More preferably, the method includes the step of attaching the non-absorbent material, whether it be a sheet or pocket, to the inside front surface of the diaper so that the non-absorbent layer is interposed between the meatus and the absorbent diaper. The non-absorbent layer or pocket may be attached to the inside front surface of the diaper by the user, such as with adhesive (which may be pre-coated onto the protective layer or pocket and provided with a release sheet) or may be pre-attached to the diaper without the need for the user to attach the sheet or pocket to the diaper.

Regardless of how the protective layer or pocket is added, the protective layer effectively prevents contact between the meatal tissue and the absorbent diaper. The flow of urine is diverted off the protective layer to surrounding absorbent diaper material so that the function of the diaper for absorbing urine is preserved. In addition, to the extent the non-absorbent protective layer or pocket contains moisturizing or therapeutic agents, the method includes treating the meatus by delivering such moisturizing or therapeutic agent to the meatus from the diaper insert. Preferably, the protective layer or pocket is replaced with each diaper change.

Additionally, the non-absorbent protective layer or pocket functions to prevent repeated abrasion of the meatus from the inner lining of the diaper. Repeated rubbing of the inner lining of the diaper against the meatus may act as an abrasive which results in irritation and scarring.

It is contemplated and within the scope of the present disclosure for the non-protective layer and/or the pocket to be impregnated with, coated with, or entirely formed of a hydrogel or the like.

Thus, the method may further constitute treating the meatus subsequent to circumcision to prevent excessive drying of the meatus and promote healing of the wound created during circumcision.

What is claimed is:

1. A diaper for improving male genital health, the diaper including a front region and a back region, the front region corresponding to a front of a male wearer, and the back region corresponding to a back of the male wearer, the diaper comprising:
an outer liquid impermeable layer having a length;
an inner absorbent layer overlying and extending for at least a portion of the length of the outer layer, the inner layer having an inner surface, a front region, a central region, and a back region, the front region of the inner layer corresponding to the front region of the diaper; and
a non-absorbent insert positioned on the inner surface of the inner layer in the proximity of the front region thereof such that when the diaper is worn by a male wearer the insert is positioned adjacent the penis of the male wearer.

2. The diaper of claim 1, further including at least one fastener connected to the outer layer for securing the back region of the diaper to the front region of the diaper when the diaper is worn by the male wearer.

3. The diaper of claim 1, wherein the insert is attached to the inside surface of the inner layer with an adhesive.

4. The diaper of claim 1, wherein the insert is selectively attachable to the inside surface of the inner layer.

5. The diaper of claim 1, wherein the insert is integrally formed into the inner layer.

6. The diaper of claim 1, further including a band of absorbent material extending about at least a portion of the perimeter of the insert.

7. The diaper of claim 1, wherein the insert is at least one of circular, rectangular, triangular, and trapezoidal in shape.

8. The diaper of claim 1, wherein the insert forms a pocket.

9. The diaper of claim 8, wherein the pocket is at least one of triangular and trapezoidal in shape.

10. The diaper of claim 8, wherein the pocket is selectively attachable to the inside surface of the inner layer.

11. The diaper of claim 8, wherein the pocket is integrally formed into the inner layer.

12. The diaper of claim 1, wherein the insert includes a moisturizing agent.

13. The diaper of claim 12 wherein the moisturizing agent includes zinc oxide.

14. The diaper of claim 12, wherein the moisturizing agent is petroleum jelly.

15. The diaper of claim 1, wherein the insert includes a therapeutic agent.

16. The diaper of claim 15, wherein the therapeutic agent is a wound healing agent.

17. A method of improving male genital health comprising the step of interposing a non-absorbent layer between the meatus and a diaper.

18. The method of claim 17, wherein the non-absorbent layer is pre-attached to the diaper.

19. The method of claim 17, wherein the non-absorbent layer is a pocket pre-attached to the diaper.

20. The method of claim 17, further comprising the step of providing a moisturizing agent associated with the non-absorbent layer.

21. The method of claim 17, further comprising the step of providing a therapeutic agent associated with the non-absorbent layer.

22. An insert for use in combination with a diaper having an inner absorbent layer, the insert comprising:
a liquid impermeable layer selectively affixable to the inner absorbent layer of the diaper, wherein the insert is positioned on the inner layer of the diaper at a location in close proximity to the meatus of a penis when the diaper is worn by a patient.

23. The insert according to claim 22, further comprising a securing means for selectively affixing the insert to the inner layer of the diaper.

24. The insert according to claim 23, wherein the securing means is selected from the group consisting of tape, adhesives, stitches, and hook and loop fasteners.

25. The insert according to claim 23, further comprising a moisturizing agent at least one of impregnated in the non-absorbent layer and coated on the non-absorbent layer.

26. The insert according to claim 23, wherein the insert is in the form of a pocket configured and dimensioned to receive the meatus of the penis therein.

27. The insert according to claim 26, wherein the insert is at least one of circular, ovular, rectangular and triangular.

28. The insert according to claim 25, further comprising an absorbent border extending at least partially around a perimeter thereof.
